(19) United States
(12) Patent Application Publication
(10) Pub. No.: US 2019/0134463 A1
(43) Pub. Date: May 9, 2019

(54) WEARABLE SYSTEM, WEARABLE DEVICE, CLOUD SERVER AND OPERATING METHOD THEREOF

(71) Applicants: LITE-ON ELECTRONICS (GUANGZHOU) LIMITED, GUANGZHOU (CN); Lite-On Technology Corporation, Taipei (TW)

(72) Inventors: Yun-Peng Ma, Taipei (TW); Pei-Hsuan Lin, Taipei (TW)

(73) Assignees: LITE-ON ELECTRONICS (GUANGZHOU) LIMITED, GUANGZHOU (CN); Lite-On Technology Corporation, Taipei (TW)

(21) Appl. No.: 15/871,081

(22) Filed: Jan. 15, 2018

(30) Foreign Application Priority Data
Nov. 3, 2017 (CN) 201711096037.9

(51) Int. Cl. A63B 24/00; H04L 29/08; G06F 1/16; A61B 5/11; A61B 5/00

(52) U.S. Cl. CPC A63B 24/0059; H04L 67/22; A63B 24/0062; A61B 5/1118; A61B 5/6802; G06F 1/163

(57) ABSTRACT

A wearable system including at least one wearable device and at least one electronic device is provided. The wearable device includes an operation program and an incentive activity program. The wearable device analyzes physiological information and/or physical activity information via the operation program. The operation program outputs a first value. The first value includes a first reward parameter for execution of the incentive activity program. The electronic device performs a transmission with the wearable device. The electronic device outputs reward data and goal setting data to a reward system. The reward system outputs a second value according to the reward data and the goal setting data. The second value includes a second reward parameter for execution of the incentive activity program. In addition, a wearable device and an operating method thereof and a cloud server are also provided.

### CROSS REFERENCE TO RELATED APPLICATION

This application claims the priority benefit of China application serial no. 201711096037.9, filed on Nov. 3, 2017. The entirety of the above-mentioned patent application is hereby incorporated by reference herein and made a part of specification.

### BACKGROUND

### Technical Field

The invention is related to a wearable system, a wearable device and a cloud server thereof and an operating method of the wearable device.

### Description of Related Art

With the development of science and technology industries, the type, function and usage of electronic devices are more and more diversified. At the same time, as electronic devices continue to become lighter and thinner, it becomes easier to carry electronic devices. The popularization of electronic devices is thus accelerated. In recent years, in order to further increase the convenience of portable electronic devices, many wearable electronic devices that can be directly worn on the user's body have been developed. The application thereof includes electronic devices worn on adults' or school-age children's bodies. Such wearable devices can track and locate the position of the children or adults and provide physiological information of the children or adults.

Therefore, one of important topics is how to promote an improvement of the lifestyle, health conditions or the like of the adults or children of school age or other ages by utilizing the electronic devices worn on their bodies.

### SUMMARY

The invention provides a wearable system, a wearable device and a cloud server thereof and an operating method of the wearable device. The wearable device can promote an improvement of a user's lifestyle and health condition and can encourage the user to achieve other planned items (such as specific learning or ability development), for example, to help the user to learn with fun.

The wearable system of the invention includes: at least one wearable device including an operation program and an incentive activity program, wherein the at least one wearable device analyzes physiological information and/or physical activity information via the operation program, and the operation program outputs a first value, wherein the first value includes a first reward parameter for execution of the incentive activity program; and at least one electronic device performing a transmission with the at least one wearable device and outputting reward data and/or goal setting data to a reward system, wherein the reward system outputs a second value according to the reward data and the goal setting data, wherein the second value includes a second reward parameter for execution of the incentive activity program.

In one embodiment of the invention, the at least one wearable device analyzes the physiological information and/or the physical activity information via an analysis engine, and the analysis engine outputs a third value, wherein the third value includes a third reward parameter for execution of the incentive activity program.

In one embodiment of the invention, the wearable system further includes: a cloud server communicating with the at least one wearable device and the at least one electronic device, wherein the reward system is disposed in the cloud server or the at least one electronic device.

In one embodiment of the invention, the analysis engine is disposed in the cloud server or the at least one wearable device.

In one embodiment of the invention, the reward system is disposed in the at least one electronic device, and the analysis engine is disposed in the at least one wearable device.

In one embodiment of the invention, the incentive activity program includes an activity. The activity includes at least one attribute. A first portion of the at least one attribute is adjusted depending on the first value, and a second portion of the at least one attribute is adjusted depending on the second value.

The wearable device of the invention includes an operation program and an incentive activity program, wherein the wearable device analyzes physiological information and/or physical activity information via the operation program, and the operation program outputs a first value, wherein the first value includes a first reward parameter for execution of the incentive activity program.

In one embodiment of the invention, the wearable device further includes an analysis engine, wherein the wearable device analyzes the physiological information and/or the physical activity information via the analysis engine, and the analysis engine outputs a third value, wherein the third value includes a third reward parameter for execution of the incentive activity program.

In one embodiment of the invention, the wearable device performs a transmission with at least one electronic device. The at least one electronic device outputs reward data and goal setting data to a reward system. The reward system outputs a second value according to the reward data and the goal setting data, wherein the second value includes a second reward parameter for execution of the incentive activity program.

In one embodiment of the invention, the wearable device communicates with a cloud server, wherein the reward system is disposed in the cloud server or the at least one electronic device.

In one embodiment of the invention, the analysis engine is disposed in the cloud server.

In one embodiment of the invention, the reward system is disposed in the at least one electronic device, and the analysis engine is disposed in the wearable device.

In one embodiment of the invention, the incentive activity program includes an activity. The activity includes at least one attribute. A first portion of the at least one attribute is adjusted depending on the first value, and a second portion of the at least one attribute is adjusted depending on the second value.

In one embodiment of the invention, the first portion includes at least one of size, shape, and vigor, and the second portion includes at least one of power, tolerance, intelligence, speed, and ornaments.

In one embodiment of the invention, the wearable device performs a competition activity and/or a collaborative activity with other wearable devices according to the incentive activity program.

The cloud server of the invention communicates with the at least one wearable device. The cloud server includes: an analysis engine receiving and analyzing physiological information and/or physical activity information from the at least one wearable device to output a value, wherein the value includes a first reward parameter for execution of the incentive activity program of the at least one wearable device.

In one embodiment of the invention, the cloud server communicating with the at least one electronic device further includes: a reward system which receives reward data and goal setting data from the at least one electronic device, and outputs another value according to the reward data and the goal setting data, wherein the another value includes a second reward parameter for execution of the incentive activity program of the at least one wearable device.

The cloud server of the invention communicates with at least one electronic device and at least one wearable device. The cloud server includes: a reward system which receives reward data and goal setting data from the at least one electronic device, and outputs a value according to the reward data and the goal setting data, wherein the value includes a reward parameter for execution of the incentive activity program of the at least one wearable device.

In one embodiment of the invention, an operating method of the wearable device is provided, wherein the wearable device includes an operation program and an incentive activity program. The operating method of the wearable device includes: analyzing physiological information and/or physical activity information and outputting a first value via the operation program, wherein the first value includes a first reward parameter for execution of the incentive activity program.

In one embodiment of the invention, the operating method of the wearable device further includes: analyzing the physiological information and/or the physical activity information and outputting a third value via an analysis engine, wherein the third value includes a third reward parameter for execution of the incentive activity program.

In one embodiment of the invention, the operating method of the wearable device further includes: performing a transmission with at least one electronic device to receive a second value from the at least one electronic device, wherein the at least one electronic device outputs reward data and goal setting data to a reward system, and the reward system outputs a second value according to the reward data and the goal setting data, wherein the second value includes a second reward parameter for execution of the incentive activity program.

In one embodiment of the invention, the operating method of the wearable device further includes: communicating with the cloud server, wherein the reward system is disposed in the cloud server or the at least one electronic device.

In one embodiment of the invention, the analysis engine is disposed in the cloud server or the wearable device.

In one embodiment of the invention, the reward system is disposed in the at least one electronic device, and the analysis engine is disposed in the wearable device.

In one embodiment of the invention, the incentive activity program includes an activity. The activity includes at least one attribute. The operating method of the wearable device further includes: adjusting a first portion of the at least one attribute depending on the first value; and/or adjusting a second portion of the at least one attribute depending on the second value.

To make the aforementioned and other features and advantages of the invention more comprehensible, several embodiments accompanied with drawings are described in detail as follows.

### BRIEF DESCRIPTION OF THE DRAWINGS

The accompanying drawings are included to provide a further understanding of the disclosure, and are incorporated in and constitute a part of this specification. The drawings illustrate exemplary embodiments of the disclosure and, together with the description, serve to explain the principles of the disclosure.

FIG. 1A illustrates a schematic diagram of a wearable system according to one embodiment of the invention.

FIG. 1B illustrates a schematic diagram of a wearable system according to an embodiment of the invention.

FIG. 1C illustrates a schematic diagram of a wearable system according to an embodiment of the invention.

FIG. 1D illustrates a schematic diagram of a wearable system according to an embodiment of the invention.

FIG. 2 illustrates a schematic diagram of the interior of a wearable device and an electronic device according to the embodiment shown in FIG. 1A.

FIG. 3 illustrates a schematic diagram of interaction between a plurality of wearable devices and electronic devices according to one embodiment of the invention.

FIG. 4 illustrates a schematic diagram of interaction between a plurality of wearable devices and electronic devices according to another embodiment of the invention.

FIG. 5 illustrates a schematic diagram of the interior of the wearable device, a cloud server, and the electronic device according to the embodiment shown in FIG. 1A.

FIG. 6 illustrates a schematic diagram of an incentive activity program and the reference data thereof according to the embodiment shown in FIG. 1A.

FIG. 7 illustrates a schematic diagram of an incentive activity program according to one embodiment of the invention.

FIG. 8 illustrates a schematic diagram of an incentive activity program according to another embodiment of the invention.

FIG. 9 illustrates a flow chart of an operating method of the wearable device according to one embodiment of the invention.

### DESCRIPTION OF THE EMBODIMENTS

In an exemplary embodiment of the invention, the user of the wearable device may be a child, for example. However, the user of the wearable device of the invention is not limited thereto. The wearable system of the invention, the wearable device of the invention, and the cloud server and the operating method thereof will be illustrated by a number of exemplary embodiments below.

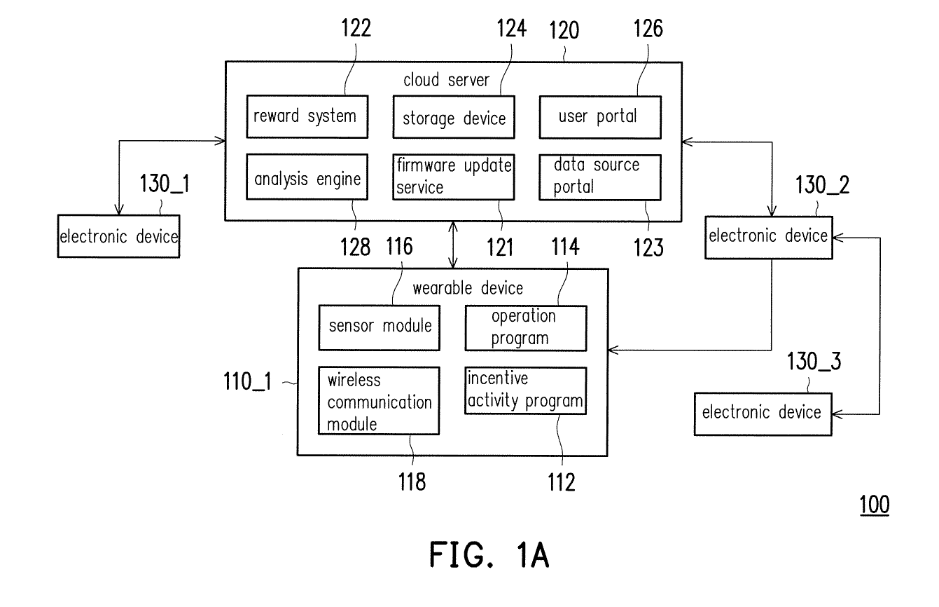

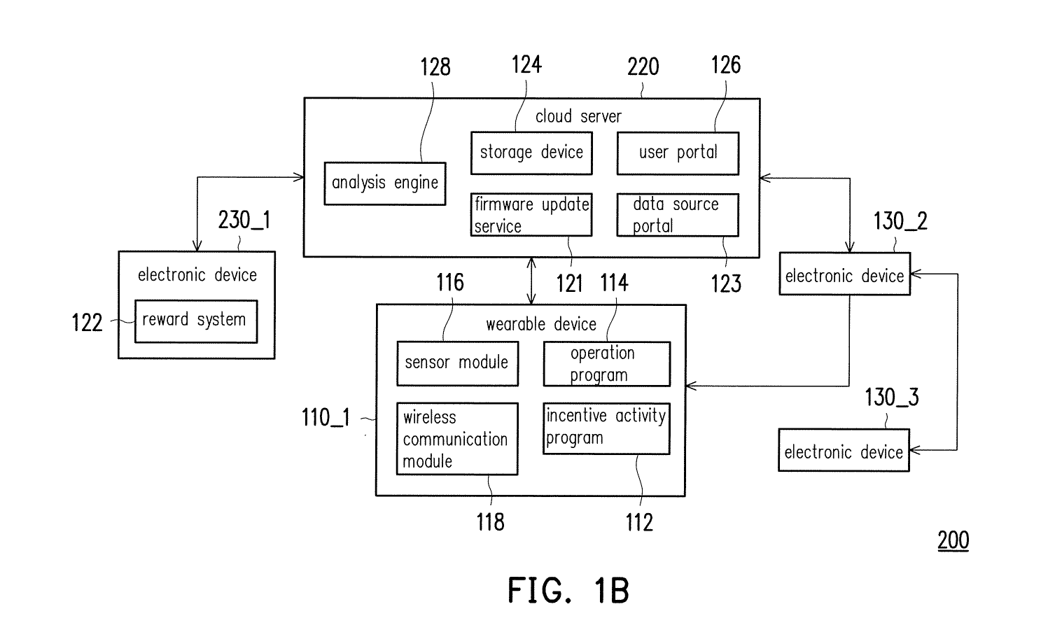

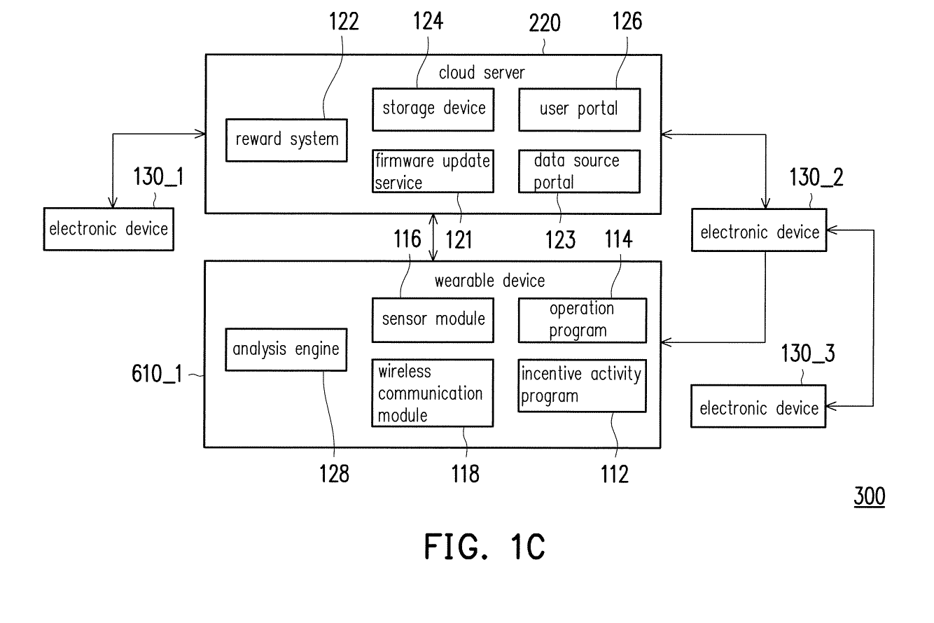

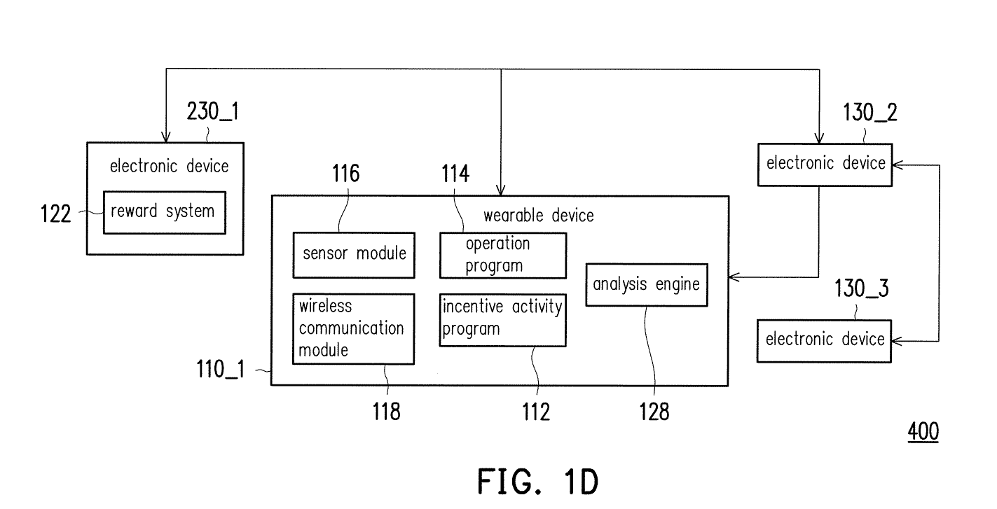

FIG. 1 A illustrates a schematic diagram of a wearable system according to one embodiment of the invention. Referring to FIG. 1A, the wearable system 100 of this embodiment includes a wearable device 110 1, a cloud server 120, and electronic devices 130_1, 130_2, and 130_3. The wearable device 110_1 and the electronic devices 130_1, 130_2, and 130_3 wirelessly communicate with the cloud server 120 to access and exchange data.

In this embodiment, the wearable device 110_1 includes an incentive activity program 112, an operation program 114, a sensor module 116, and a wireless communication module 118. The user of the wearable device 110_1 includes, for example, adults or school-age children (students). The sensor module 116 includes at least one sensor such as an accelerometer (G sensor), a Gyro sensor, a magnetic sensor (M sensor) and the like. In this embodiment, the sensor module 116 detects various physiological information and/or physical activity information, wherein the physiological information includes information such as sleep time, heart rate and the like, and the physical activity information includes sport information such as the number of walking steps, running, bike riding, and the like and information such as calories burned, fitness index, and the like. However, the types of the sensors, the physiological information, and the physical activity information are not limited thereto.

In this embodiment, the wearable device 110_1 utilizes a wireless communication module 118 to wireless communicate with the cloud server 120 and the electronic devices 130_1, 130_2, and 130_3 to access and exchange data. The wireless communication module 118 includes, for example, fourth generation (4G) mobile communication technology module, wireless LAN (Wi-Fi) technology module or Bluetooth communication technology module. In this embodiment, the incentive activity program 112 includes, for example, a game program such as a pet game program or a racing game program. In an embodiment, the incentive activity program 112 may also be other various activity programs, for example, a health program by which various health improvement courses of a health center can be provided. However, the types of the wireless communication modules and the incentive activity programs of the invention are not limited thereto.

In this embodiment, the cloud server 120 includes a firmware update service 121, a reward system 122, a data source portal 123, a storage device 124, a user portal 126, and an analysis engine 128. In this embodiment, the operation program 114 includes, for example, an operation program for detecting sport and health, wherein the operational analysis of the physiological information and/or physical activity information can be executed by the operation program 114. In this embodiment, the result of the operational analysis may include a first value. The first value includes a first reward parameter such as a health reward parameter, and the parameter may be determined by or presented as a numerical value such as a point, a score, or a weight. In this embodiment, the analysis engine 128 performs, for example, the operational analysis or deep learning for the physiological information and/or the physical activity information obtained from the wearable device 110_1 in order to analyze and predict the behavior and/or the living habit of the user, and output the results of the analysis for long-term tracking. The firmware update service 121 includes, for example, a Firmware Over-The-Air (FOTA) service, which is mobile device software management technology capable of upgrading or updating the firmware/software of the wearable device 110_1 online with a wireless communication, such as updating the incentive activity program 112. However, the firmware update service of the invention is not limited thereto. In an embodiment, the analysis engine 128 may also be integrated with the operation program 114 into an operational analysis program disposed in the wearable system 100 or the cloud server 120.

In this embodiment, the data source portal 123 serves as, for example, an output/input interface and is used to transmit first data with the wearable device 110_1. The first data includes, but not limited to, a reward parameter (e.g., a reward point), incentive activity data, goal setting data, and various physiological information and/or physical activity information of the user of the wearable device 110_1, wherein the physiological information and/or the physical activity information are as described above. The user portal 126 serves as, for example, another output/input interface, and is used to transmit second data with the electronic devices 130_1, 130_2, and 130_3. The second data includes, but not limited to, the reward data, the goal setting data, the incentive activity data and/or the analysis result provided by the analysis engine 128. In an embodiment, the first data and the second data may also be directly transmitted from the electronic devices 130_1, 130_2, and 130_3 to the wearable device 110_1 without being passed through the cloud server 120. In this embodiment, a storage device 124 is used to store the data received by the cloud server 120 from other devices, and may also include a deep learning database.

In an embodiment, the electronic devices 130_1, 130_2, and 130_3 are, for example, portable electronic devices such as a mobile phone, a tablet, a notebook, and a smart watch. In an embodiment, the electronic devices 130_1, 130_2, and 130_3 are, for example, non-portable electronic devices such as a desktop computer. The types of the electronic devices of the invention are not limited thereto, and the types of each of the electronic devices may be the same or different.

In this embodiment, the electronic device 130_1, for example, stores the reward data and the goal setting data in the storage device 124 via the user portal 126 and then the data are transmitted to the reward system 122 of the cloud server 120. The electronic device 130_1 may also receive the analysis result of the analysis engine 128 via the user portal 126. The reward system 122 calculates and outputs a second value according to the reward data and the goal setting data. The second value includes a second reward parameter such as a reward point. The reward data and the goal setting data include, for example, one or more predetermined rules. For example, the goal is set as exercise for half an hour daily, and the reward data may include one sport medals obtained at a certain sport due to the performance and one reward point issued for 3 sport medals. The reward system 122 may calculate the second reward parameter (e.g., the reward point) based on a child's learning condition or performance in the school. Next, the reward system 122 saves the reward parameter into the storage device 124, and then the reward parameter is transmitted to the incentive activity program 112 of the wearable device 110_1 via the data source portal 123. The activity attributes of the incentive activity program 112 may vary depending on the first value or the first reward parameter (e.g., the health reward parameter) and/or the second value or the second reward parameter (e.g., the reward point). Therefore, the reward point or the health reward parameter serves as incentives to promote or lead the user of the wearable device 110_1 (e.g., a child) to change living habits, health conditions (e.g., in the way of exercise) or school learning to obtain good conditions or attributes of an incentive activity for engaging in the incentive activity, for example, to obtain good conditions of a game for engaging in the game (taking a game program as an example). In addition to being entertaining, the wearable device is also learning-inspiring and can help the child shape good habits and forge a strong body (such as active exercise and adequate sleep). In an embodiment, the reward data and the goal setting data can also be set by the user of the electronic devices 130_2 and 130_3 (i.e., the child's father and/or mother). However, the invention is not limited thereto. In addition, the user of the wearable device 110_1 may be prompted to adjust his/her own characteristics such as living habits, health conditions (e.g., in the way of exercise) or school learning by feedback of information such as the second reward points or the health reward parameters. Therefore, the wearable device 110_1 is helpful for the user to grow healthily and develop good living habits or achieve learning goals. That is, the user of the wearable device 110_1 obtains the first value (e.g., the health reward parameter) and/or the second value (e.g., the reward point) by adjusting his/her own characteristics such as living habits, health conditions (in the way of exercise) or learning.

FIG. 1B illustrates a schematic diagram of a wearable system according to another embodiment of the invention. Referring to FIG. 1B, the wearable system 200 of this embodiment is similar to the wearable system 100 of the embodiment of FIG. 1A. The main difference therebetween is that, for example, the electronic device 230_1 includes a reward system 122. In this embodiment, the reward system 122 can be miniaturized and then placed in the electronic device 230_1.

FIG. 1 C illustrates a schematic diagram of a wearable system according to another embodiment of the invention. Referring to FIG. 1C, the wearable system 300 of this embodiment is similar to the wearable system 100 of the embodiment of FIG. 1A. The main difference therebetween is that, for example, the wearable device 110_1 includes an analysis engine 128. In this embodiment, the analysis engine 128 can be miniaturized and then placed in the wearable device 110_1.

FIG. 1D illustrates a schematic diagram of a wearable system according to another embodiment of the invention. Referring to FIG. 1D, the wearable system 400 of this embodiment is similar to the wearable system 200 of the embodiments of FIG. 1B or the wearable system 300 of the embodiments of FIG. 1C. The main difference therebetween is that, for example, the wearable device 400 does not include a cloud server. In this embodiment, the children wearable device 110_1 exchanges various data with the electronic devices 230_1, 130_2, and 130_3 directly without being through the cloud server.

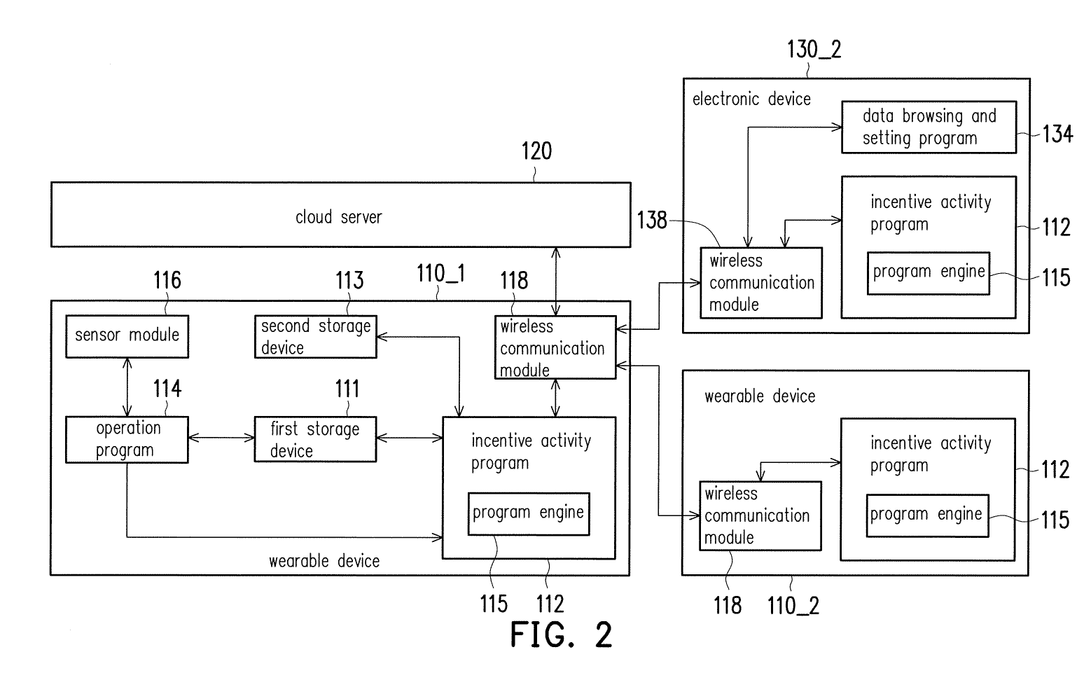

FIG. 2 illustrates a schematic diagram of the interior of the wearable device and the electronic device according to the embodiment of FIG. 1A. Referring to FIG. 1A and FIG. 2, in this embodiment, the wearable device 110_1 further includes a first storage device 111 and a second storage device 113. The incentive activity program 112 includes a program engine 115. The program engine 115 is a system for running the incentive activity program. The first storage device 111 is used to store the physiological information and/or the physical activity information of the user of the wearable device 110_1. The second storage device 113 is used to store the incentive activity data of the incentive activity program 112. For example, when the incentive activity program 112 is a game program, the incentive activity data includes, for example, various information regarding the game which the user of the wearable device 110_1 plays, for example, including, but not limited to, a value of a graphic file or an attribute. The program engine 115 may also access the physiological information and/or physical activity information of the user from the first storage device 111 for being used as a parameter of the incentive activity program 112. The invention does not limit the number (including only one), the type and the storage method of the storage device of the wearable device 110_1.

In this embodiment, the operation program 114 receives sensed data from the sensor module 116, i.e., physiological information and/or physical activity information. The physiological information and physical activity information are, for example, the physiological information and physical activity information as described above. In addition, the operation program 114 may also calculate operation control events or data corresponding to the incentive activity program 112 according to the user's gesture (including clicks, shakes and other gestures).

The operation program 114 stores the physiological information and physical activity information and the operation result thereof in the first storage device 111. Taking a pet game program as an example, a child may use a finger to touch a touch screen of the wearable device 110_1 to confirm the status of a pet, such as the pet's vitality or growth status, or to interact with the pet. In an embodiment, the operation program 114 may obtain the first value according to the goal setting data, the physiological information and/or the physical activity information, and then the incentive activity program 112 is executed according to the first value and/or other reward parameters. The goal setting data may be such as a goal parameter and predetermined by the device or provided by the teacher or parent. The goal setting data may be stored in the wearable device 110_1 or the reward system 122.

In this embodiment, taking the electronic device 130_2 as an example, the electronic device 130_2 includes the incentive activity program 112 and a wireless communication module 138. In this embodiment, the child's father or mother can also interact with the child through the incentive activity program 112 of the electronic device 130_2. In this embodiment, the electronic device 130_2 utilizes the wireless communication module 138 to perform a wireless communication with the cloud server 120, the wearable device 110 1, and the electronic devices 130_1 and 130_3 for data access and exchange. The wireless communication module 138 includes, for example, fourth generation mobile communication technology module, wireless LAN technology module or Bluetooth communication technology module. The internal structures of the electronic devices 130_1 and 130_3 can be analogized by the electronic device 1302.

In this embodiment, FIG. 2 further illustrates the wearable device 110_2, indicating that the wearable system 100 may include one or more the wearable devices. In the wearable system 100, the wearable device 110_1 may also interact and communicate with another wearable device 110_2 in addition to interacting with the wearable devices 130_1, 130_2, and 130_3. The internal structure of the wearable device 110_2 can be analogized by the wearable device 110_1.

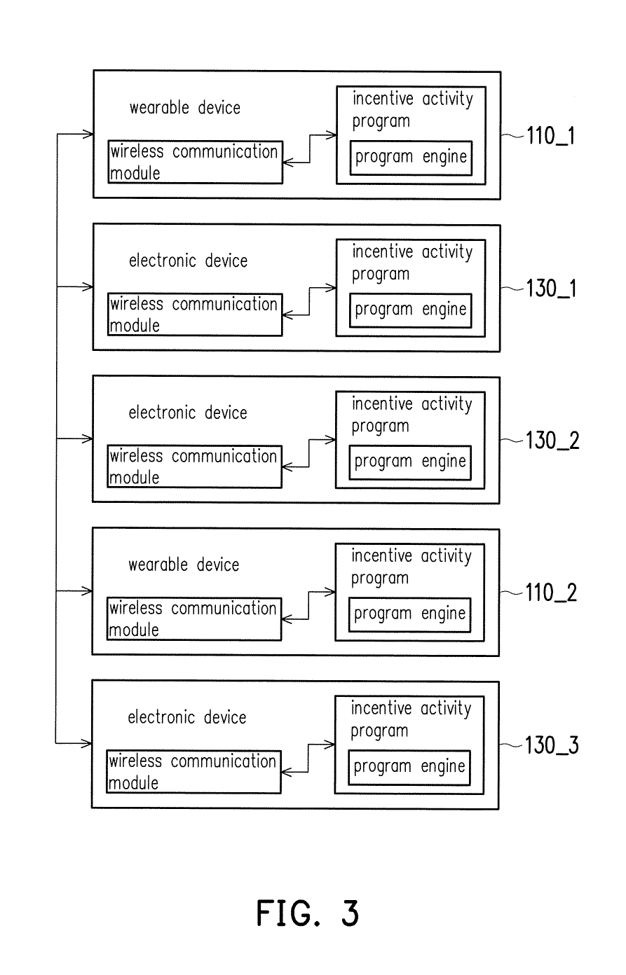

FIG. 3 illustrates a schematic diagram of the interaction between a plurality of the wearable devices and the electronic devices according to one embodiment of the invention. Referring to FIG. 3, in this embodiment, the incentive activity program is, for example, a racing game program, but may also be a pet game or other programs without limitation. The wearable devices 110_1 and 110_2 and the electronic devices 130_1, 130_2 and 130_3 can compete in the racing game. In an embodiment, the game mode may also be performed by the interactions between the plurality of the wearable devices without the participations of the electronic devices 130_1, 130_2, and 130_3. However, the invention is not limited thereto.

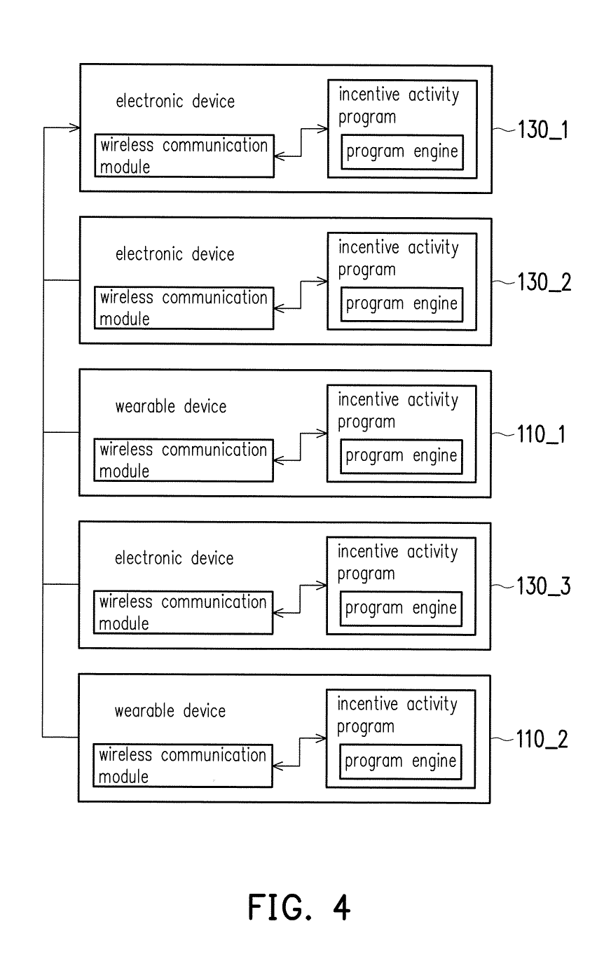

FIG. 4 illustrates a schematic diagram of the interaction between a plurality of the wearable devices and the electronic devices according to another embodiment of the invention. Referring to FIG. 4, in this embodiment, the incentive activity program is, for example, a racing game program, but may also be a pet game or other programs without limitation. The wearable devices 110_1 and 110_2 and the electronic devices 130_1, 130_2 and 130_3 can cooperate in the racing game to complete a team mission. In an embodiment, the game mode may also be performed by the interactions between the plurality of the wearable devices without the participations of the electronic devices 130_1, 130_2, and 130_3. However, the invention is not limited thereto.

Thus, in the embodiments of FIGS. 3 and 4, the wearable devices 110_1 and 110_2 and the electronic devices 130_1, 130_2, and 130_3 may exchange the interactive data via the cloud server 120, or the wearable devices 110_1 and 110_2 and the electronic devices 130_1, 130_2, and 130_3 exchange the interactive data directly. The manner for exchanging the interactive data of the invention is not limited. The competitive activities (FIG. 3) and/or the collaborative activities (FIG. 4) of the incentive activity program can be executed according to the interactive data. The interactive data includes the incentive activity program 112 and/or the incentive activity data.

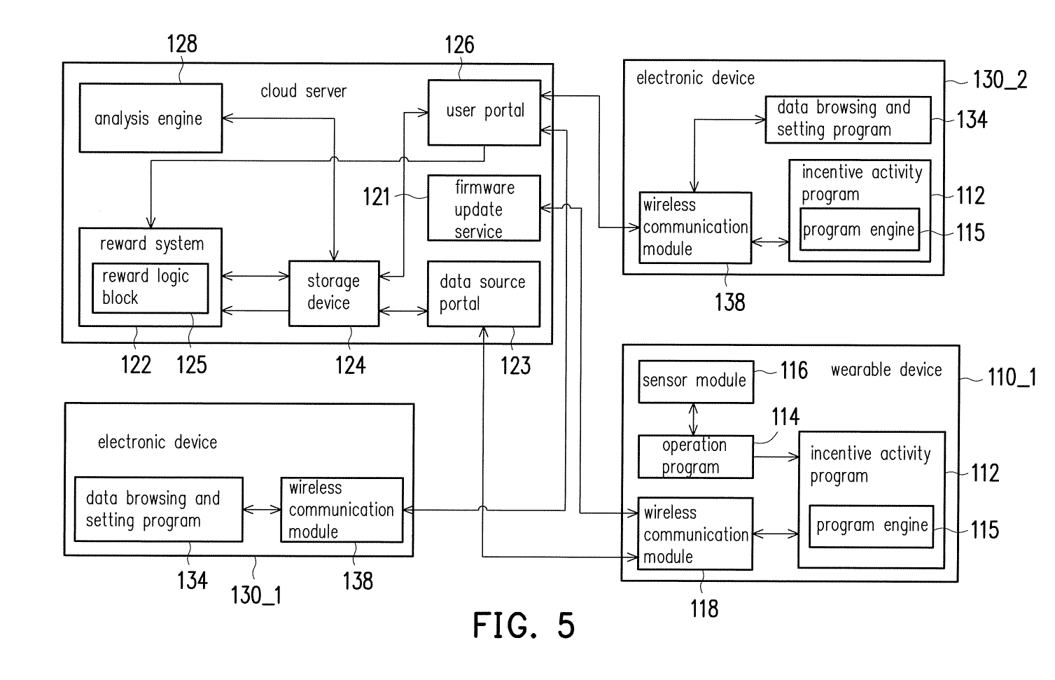

FIG. 5 illustrates a schematic diagram of the interior of the wearable device, the cloud server, and the electronic device according to the embodiment shown in FIG. 1A. Referring to FIG. 1A and FIG. 5, the reward system 122 of this embodiment includes a reward logic block 125. In this embodiment, the reward data, the goal setting data and the incentive activity data which are received from the electronic devices 130_1, 130_2, and 130_3 by the user portal 126 may be transmitted to the storage device 124 and stored therein. The reward logic block 125 then accesses the reward data and the goal setting data from the storage device 124. Alternatively, the data are transmitted from the user portal 126 to the reward logic block 125 directly. In this embodiment, the reward logic block 125 performs operations according to the reward data and the goal setting data, and converts the operation result into a second value, which includes a second reward parameter (e.g., a reward point). In this embodiment, the electronic devices 130_1, 130_2, and 130_3 may also receive the analysis result of the analysis engine 128 via the user portal 126. In this embodiment, the physiological information and/or the physical activity information and an incentive activity data received from the wearable device 110_1 by the data source portal 123 may be transmitted to the storage device 124 and stored therein. In this embodiment, the analysis engine 128 analyzes the various data received from the wearable device 110_1 by deep learning. The produced analysis result may also be transmitted to the storage device 124 and stored therein. The reward logic block 125 accesses the analysis result to calculate a third value which may include a third reward parameter (e.g., a reward point). For example, the reward logic block 125 gives one reward point to the user who continuously runs for half an hour daily for one month. The incentive activity program 112 of the wearable device 110_1 can read the second and the third reward points.

In this embodiment, taking the electronic devices 130_1 and 130_2 as examples, each of the electronic devices 130_1 and 130_2 includes a data browsing and setting program 134. In this embodiment, the child's father or mother can know the status of the incentive activity program 112 of the wearable device 110_1 through the data browsing and setting program 134. For example, the child's father or mother can know whether the child plays the game at an inappropriate time and can adjust the setting accordingly.

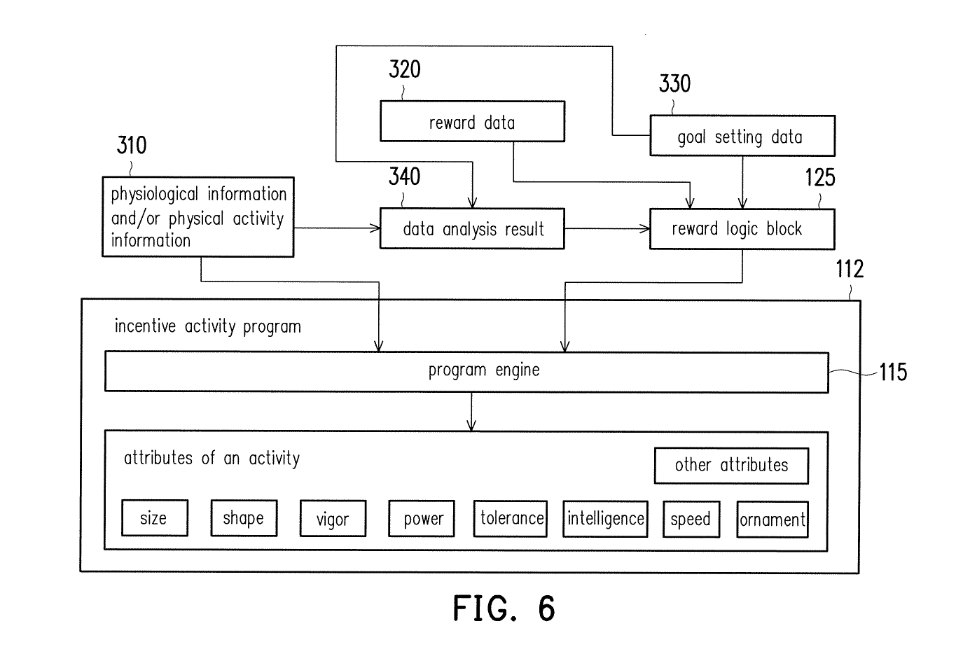

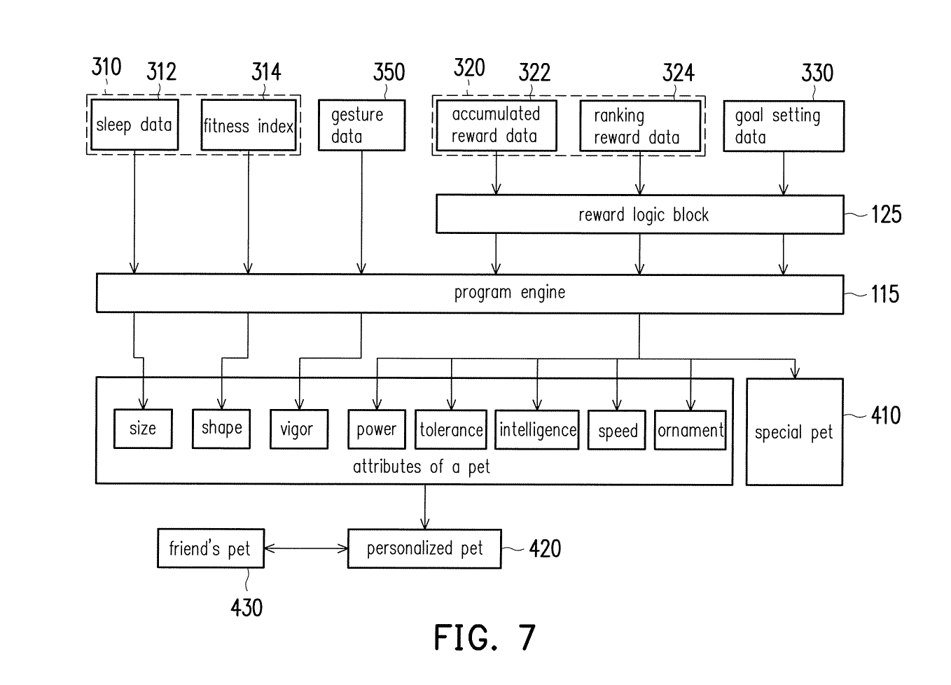

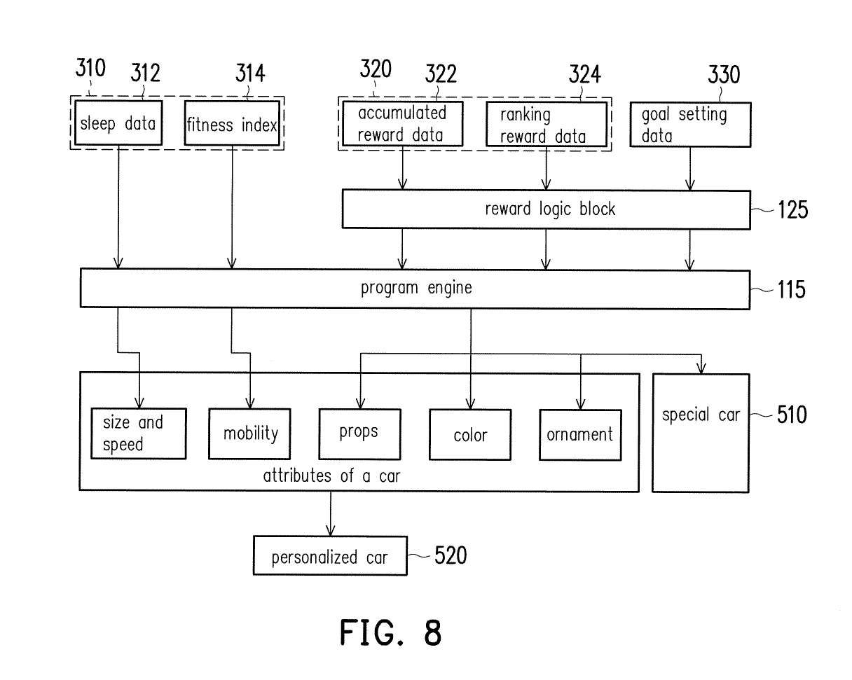

FIG. 6 illustrates a schematic diagram of the incentive activity program and the reference data thereof according to the embodiment shown in FIG. 1A. Referring to FIG. 1A and FIG. 6, in this embodiment, the analysis engine 128, for example, produces the analysis result 340 by deep learning the physiological information and/or physical activity information 310 and combining the goal setting data 330. The analysis result 340 is then transmitted to the storage device 124 for being accessed by the reward logic block 125. In this embodiment, the reward logic block 125 performs operations according to various reference data such as the reward data 320 and the goal setting data 330 (e.g., the goal parameter), and transmits the operation result including the second value (e.g., the reward point) to the program engine 115. In this embodiment, the incentive activity program 112 includes an activity. The activity includes at least one attribute. In this embodiment, the program engine 115 drives the incentive activity program 112 to display the attributes of the incentive activity according to the physiological information and/or the physical activity information 310 and the data received from the reward logic block 125. In this embodiment, the manner of how the program engine 115 drives the incentive activity program 112 to display the attributes of the activity may refer to the following embodiments in FIG. 7 and FIG. 8. In an embodiment, depending on the contents of the incentive activity program 112, the attributes may be presented as options. In addition, the incentive activity program 112 may include a plurality of activities or the wearable devices 110_1 may also have a plurality of incentive activity programs 112.

FIG. 7 illustrates a schematic diagram of an incentive activity program according to one embodiment of the invention. Referring to FIG. 7, the incentive activity program 112 in this embodiment is, for example, a pet game program. In this embodiment, the physiological information and/or physical activity information 310 include sleep data 312 and a fitness index 314. For example, the sleep data 312 may affect the size of the pet, such as the height and weight of the pet. The fitness index 314, for example, affects the shape of the pet, such as the body shape of the pet. In this embodiment, the child may use a finger to touch the touch screen of the wearable device 110_1 to generate the gesture data 350 so as to confirm the condition of the pet, such as the vigor of the pet, by using the touch gesture.

In this embodiment, the reward data 320 includes, for example, accumulated reward data 322 and ranking reward data 324. The accumulated reward data 322 includes, for example, a predetermined first rule. The reward points corresponding to the child's learning status in a class or learning status in a group may be calculated according to the first rule. The ranking reward data 324 includes, for example, a second predetermined rule. The corresponding reward points for the rank of the child's competition or school grades may also be calculated according to the second rule. The goal setting data 330 includes, for example, a predetermined third rule. The corresponding reward points may be calculated according to whether the child's learning goals are achieved and the third rule. In this embodiment, the first rule, the second rule, and the third rule may be the same or different. In this embodiment, the reward logic block 125 may calculate the reward points according to the accumulated reward data 322, the ranking reward data 324, and the goal setting data 330, and output the reward points to the program engine 115. The reward points can be stored in the pet game program. The child may enhance the attributes of the pet (such as the power, tolerance, speed, and intelligence) and buy ornaments so as to create a personalized pet 420 by consuming the reward points. Alternatively, the child may obtain the pet with special attributes 410 by consuming the reward points. In this embodiment, the child may also use his/her personalized pet to battle with his/her friend's pet 430 to being entertaining.

In this embodiment, the pet is part of the game to attract the child to own, raise, play with, and interact with his/her pet. The pet can be a cat, a dog, fish, or other animals or characters (such as Celebi in Pokemon). In this embodiment, the incentive activity program 112 includes at least one attribute. A first portion of the attributes includes size, shape, and vigor. In this embodiment, the attributes such as size, shape, and vigor are adjustable according to the first value (e.g., the health parameter). A second portion of the attributes includes at least one of power, tolerance, intelligence, speed, and ornaments. In this embodiment, the attributes such as power, tolerance, intelligence, speed, and ornaments are adjustable according to the second value (for example, the reward point). Various attributes of the game program are further explained in the following. In an embodiment, the size of the pet may be designed to have different sizes at different stages of growth. For example, the nutrients for the growth of the pet to a next stage come from the sleep data 312 of the user of the wearable device 110_1. The sleep data 312 may be converted into the corresponding health reward parameter (e.g., points) to affect the size of the pet. For example, the more the health points, the larger the size of the pet. In an embodiment, the shape of the pet may be designed to have different shapes at different stages of health according to the fitness index 314 of the user of the wearable device 110_1, for example. The fitness index 314 may be converted into the corresponding health reward parameter (e.g., points) to affect the shape of the pet. For example, the more balanced the fitness index 314, the more balanced the shape of the pet. In order to maintain the shape of the pet, the user of the wearable device 110_1 must continuously and regularly exercise to keep the fitness index 314 balanced. In an embodiment, the vigor of the pet may be designed to have different vigor at different stages of energy. The user of the wearable device 110_1 may interact or play with the pet employing different gestures. The pet looks cheerful or happy when the user interacts or plays games with the pet. The more the user interacts with the pet, the more vigor the pet will have. In an embodiment, the vigor of the pet may also be increased by consuming the reward points. In an embodiment, different pets may be designed to have different ornaments to promote the user of the wearable device 110_1 to gain the reward. For example, the user must perform well in class to get more reward points to exchange for different ornaments. In an embodiment, the first portion of the attributes cannot be adjusted unless the first value is not less than a threshold value.

For example, the size of the pet will not change unless the health points corresponding to the attribute of size is not less than the threshold value. Therefore, it is helpful to encourage the child to improve the living habits and/or physical health activities which make the first value (i.e., the health reward parameter) equal to or greater than the threshold value. 100681 In an embodiment, the pet is designed to have the characteristic of speed which serves as part of a battle ability, and a higher speed represents a better advantage in a pet battle game. To increase the speed of the pet, the user of the wearable device 110_1 must come out in front in the running grades to obtain the points for increasing the speed. In an embodiment, the pet is designed to have the characteristic of power which serves as part of the battle ability, and a stronger power represents a higher chance of victory in the pet battle game. To increase the power of the pet, the user of the wearable device 110_1 must come out in front in the sport requiring power to obtain the points for increasing the power. In an embodiment, the pet is designed to have the characteristic of tolerance which serves as part of the battle ability, and a more robust tolerance represents a higher chance of surviving in the pet battle game. To increase the tolerance of the pet, the user of the wearable device 110_1 must continue to reach the daily predetermined goal to obtain the points for increasing the tolerance. In an embodiment, the pet is designed to have the characteristic of intelligence which serves as part of the battle ability, and a higher intelligence represents the ability to use more skills in a pet battle game. To increase the intelligence of the pet, the user of the wearable device 110_1 must come out in front in the grades of the reward items of the school to obtain the points for increasing the intelligence. The attributes of the game of the invention are not limited to the attributes illustrated above. The game or activity concept suitable for the incentive activity of the invention is also not limited thereto. The connection between any other game or activity concepts and the data from the wearable device 110_1 or the cloud server 120 may also be regarded as an extension of the game or activity suitable for the invention.

FIG. 8 illustrates a schematic diagram of an incentive activity program according to another embodiment of the invention. Referring to FIG. 8, the incentive activity program 112 in this embodiment is, for example, a racing game program. In this embodiment, the incentive activity program 112 includes at least one attribute. The first portion of the attributes includes size, speed and mobility. In this embodiment, the attributes such as size, speed and mobility are adjustable according to the first value (e.g., the health parameter). The second portion of the attributes includes at least one of props, color and ornament. In this embodiment, attributes such as props, color and ornament are adjustable according to the second value (for example, the reward point). In this embodiment, the physiological information and/or the physical activity information 310 include the sleep data 312 and the fitness index 314. The sleep data 312 may be converted into the corresponding health reward parameter (e.g., points) to affect the size and the speed of the car. For example, the more the health points, the larger the size of the car and the faster the speed of the car. The fitness index 314 may be converted into the corresponding health reward parameter (e.g., points) to affect the mobility of the car. For example, the more the health points, the higher the mobility of the car.

In this embodiment, the reward logic block 125 may calculate the reward points according to the accumulated reward data 322, the ranking reward data 324, and the goal setting data 330, and output the reward points to the program engine 115. The reward points can be stored in the game program. The child may change the attributes of the car (such as color) and buy game props and ornaments to create a personalized car 520 by consuming the reward points. Alternatively, the child may obtain a car with special attributes 510 by consuming the reward points. The incentive activity programs of FIG. 7 and FIG. 8 are only used for illustration, and are not intended to limit the invention.

In the exemplary embodiment of the invention, the functional blocks of the wearable devices 110_1 and 110_2, the cloud server 120, and the electronic devices 130_1, 130_2, and 130_3 are either implemented by being formed in the logic circuit (hardware) such as in an integrated circuit (IC chip), or by software via the central processing unit (CPU).

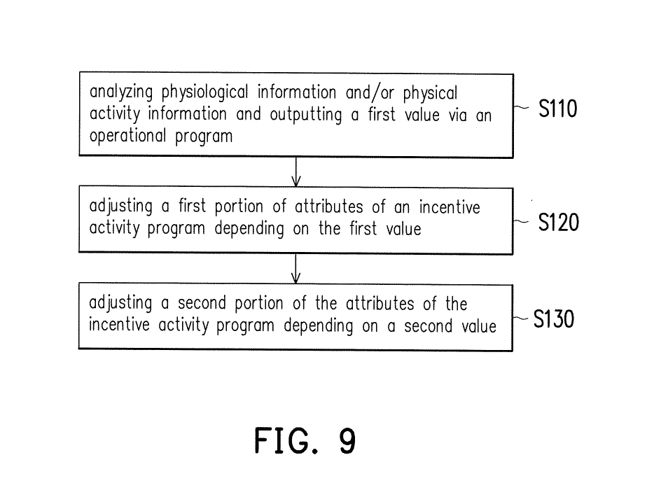

FIG. 9 illustrates a flow chart of an operating method of the wearable device according to one embodiment of the invention. Referring to FIG. 9, in this embodiment, in step S110, the operational program analyzes the physiological information and/or the physical activity information and outputs the first value, wherein the first value includes a reward parameter for execution of the incentive activity program. In step S120, a first portion of the attributes of the incentive activity program is adjustable depending on the first value. In step S130, a second portion of the attributes of the incentive activity program is adjustable depending on the second value. In an embodiment, the operating method of the wearable device includes outputting the reward data and goal setting data to a reward system. The reward system outputs the second value according to the reward data and the goal setting data, wherein the second value includes a reward parameter for execution by the incentive activity program. In addition, the embodiments shown in FIG. 1A to FIG. 8 provide sufficient teaching, suggestions and implementation instructions regarding the operation method of the wearable device according to the embodiment of the invention, and therefore the descriptions are not repeated herein.

In summary, in the exemplary embodiment of the invention, the user of the wearable device (e.g., a child) is encouraged to change living habits, health conditions (e.g., in the way of exercise) or learning status to obtain corresponding good conditions of an incentive activity for engaging in the incentive activity by quantifying the assessments for the physiological information and/or physical activity information or learning as a reward parameter. The wearable device is helpful to promote an improvement of the lifestyle and health condition of the user of the wearable device and encourage the user to achieve specific learning or ability development.

Although the embodiments are already disclosed as above, these embodiments should not be construed as limitations on the scope of the invention. It will be apparent to those skilled in the art that various modifications and variations can be made to the disclosed embodiments without departing from the scope or spirit of this invention. In view of the foregoing, it is intended that the invention covers modifications and variations provided that they fall within the scope of the following claims and their equivalents.

What is claimed is:

1. A wearable system comprising:
at least one wearable device comprising an operation program and an incentive activity program, wherein the at least one wearable device analyzes physiological information and/or physical activity information via the operation program, and the operation program outputs a first value, wherein the first value comprises a first reward parameter for execution of the incentive activity program; and
at least one electronic device performing a transmission with the at least one wearable device and outputting reward data and/or goal setting data to a reward system, wherein the reward system outputs a second value according to the reward data and the goal setting data, wherein the second value comprises a second reward parameter for execution of the incentive activity program.

2. The wearable system according to claim 1, wherein the at least one wearable device analyzes the physiological information and/or the physical activity information via an analysis engine, and the analysis engine outputs a third value, wherein the third value comprises a third reward parameter for execution of the incentive activity program.

3. The wearable system according to claim 2, further comprising:
a cloud server communicating with the at least one wearable device and the at least one electronic device, wherein the reward system is disposed in the cloud server or the at least one electronic device.

4. The wearable system according to claim 3, wherein the analysis engine is disposed in the cloud server or the at least one wearable device.

5. The wearable system according to claim 2, wherein the reward system is disposed in the at least one electronic device, and the analysis engine is disposed in the at least one wearable device.

6. The wearable system according to claim 1, wherein the incentive activity program comprises an activity, and the activity comprises at least one attribute, wherein a first portion of the at least one attribute is adjusted depending on the first value, and a second portion of the at least one attribute is adjusted depending on the second value.

7. A wearable device comprising an operation program and an incentive activity program, wherein the wearable device analyzes physiological information and/or physical activity information via the operation program, and the operation program outputs a first value, wherein the first value comprises a first reward parameter for execution of the incentive activity program.

8. The wearable device according to claim 7, further comprising an analysis engine, wherein the wearable device analyzes the physiological information and/or the physical activity information via the analysis engine, and the analysis engine outputs a third value, wherein the third value comprises a third reward parameter for execution of the incentive activity program.

9. The wearable device according to claim 8, wherein the wearable device performs a transmission with at least one electronic device, the at least one electronic device outputs reward data and goal setting data to a reward system, and the reward system outputs a second value according to the reward data and the goal setting data, wherein the second value comprises a second reward parameter for execution of the incentive activity program.

10. The wearable device according to claim 9, wherein the wearable device communicates with a cloud server, wherein the reward system is disposed in the cloud server or the at least one electronic device.

11. The wearable device according to claim 10, wherein the analysis engine is disposed in the cloud server.

12. The wearable device according to claim 9, wherein the reward system is disposed in the at least one electronic device, and the analysis engine is disposed in the wearable device.

13. The wearable device according to claim 7, wherein the wearable device performs a transmission with at least one electronic device, the at least one electronic device outputs reward data and goal setting data to a reward system, and the reward system outputs a second value according to the reward data and the goal setting data, wherein the second value comprises a second reward parameter for execution of the incentive activity program.

14. The wearable device according to claim 7, wherein the incentive activity program comprises an activity, and the activity comprises at least one attribute, wherein a first portion of the at least one attribute is adjusted depending on the first value, and a second portion of the at least one attribute is adjusted depending on the second value.

15. The wearable device according to claim 14, wherein the first portion comprises at least one of size, shape, and vigor, and the second portion comprises at least one of power, tolerance, intelligence, speed, and ornament.

16. The wearable device according to claim 7, wherein the wearable device performs a competition activity and/or a collaborative activity with other wearable devices according to the incentive activity program.

17. A cloud server communicating with at least one wearable device and comprising:
an analysis engine receiving and analyzing physiological information and/or physical activity information from the at least one wearable device to output a value, wherein the value comprises a first reward parameter for execution of an incentive activity program of the at least one wearable device.

18. The cloud server according to claim 17, further communicating with at least one electronic device and comprising:
a reward system receiving reward data and goal setting data from the at least one electronic device, and outputting another value according to the reward data and the goal setting data, wherein the another value comprises a second reward parameter for execution of the incentive activity program of the at least one wearable device.

19. A cloud server, communicating with at least one electronic device and at least one wearable device and comprising:
a reward system receiving reward data and goal setting data from the at least one electronic device, and outputting a value according to the reward data and the goal setting data, wherein the value comprises a reward parameter for execution of an incentive activity program of the at least one wearable device.

20. An operating method of a wearable device, wherein the wearable device comprises an operation program and an incentive activity program, the operating method of the wearable device comprising:
analyzing physiological information and/or physical activity information and outputting a first value via the operation program, wherein the first value comprises a first reward parameter for execution of the incentive activity program.

21. The operating method of the wearable device according to claim 20, further comprising:
analyzing the physiological information and/or the physical activity information and outputting a third value via an analysis engine, wherein the third value comprises a third reward parameter for execution of the incentive activity program.

22. The operating method of the wearable device according to claim 21, further comprising:
performing a transmission with at least one electronic device to receive a second value from the at least one electronic device, wherein the at least one electronic device outputs reward data and goal setting data to a reward system, and the reward system outputs a second value according to the reward data and the goal setting data, wherein the second value comprises a second reward parameter for execution of the incentive activity program.

23. The operating method of the wearable device according to claim 22, further comprising:
communicating with a cloud server, wherein the reward system is disposed in the cloud server or the at least one electronic device.

24. The operating method of the wearable device according to claim 23, wherein the analysis engine is disposed in the cloud server or the wearable device.

25. The operating method of the wearable device according to claim 24, wherein the reward system is disposed in the at least one electronic device, and the analysis engine is disposed in the wearable device.

26. The operating method of the wearable device according to claim 22, wherein the incentive activity program comprises an activity, and the activity comprises at least one attribute, the operating method of the wearable device further comprising:
adjusting a first portion of the at least one attribute depending on the first value; and/or
adjusting a second portion of the at least one attribute depending on the second value.
